(19) United States
(12) Patent Application Publication
(10) Pub. No.: US 2012/0232158 A1
(43) Pub. Date: Sep. 13, 2012

(54) METHODS OF TREATING HYPERACIDIC DISORDERS

(75) Inventors: John Peter Geibel, Branford, CT (US); Steven C. Hebert, Plantation, FL (US); Patricia R. Hebert, Plantation, FL (US); David Martin, San Francisco, CA (US)

(73) Assignees: Yale Univeristy; Amgen Inc

(21) Appl. No.: 12/919,440

(22) PCT Filed: Mar. 3, 2009
(86) PCT No.: PCT/US09/01381

§ 371 Date: Sep. 26, 2011

Related U.S. Application Data
(60) Provisional application No. 61/068,065, filed on Mar. 3, 2008.

(51) Int. Cl. A61K 31/277; A61P 1/00; A61P 29/00; A61P 11/06; A61P 11/14; A61P 1/04; A61P 1/08

(52) U.S. Cl. 514/653

(57) ABSTRACT

The present invention relates to methods for treating or preventing hyperacidic disorders such as GERD or NERD using calcium receptor active compounds.

### FIELD OF THE INVENTION

This invention relates generally to the field of medicine and, more specifically, to methods for treating or preventing of hyperacidic disorders such as GERD or NERD.

### BACKGROUND OF THE INVENTION

Over 30 million people suffer from symptoms of acid related diseases per year with the numbers increasing yearly. Gastroesophageal reflux disease (GERD) is a spectrum of diseases usually producing symptoms of heartburn and acid regurgitation. Most patients with non-erosive esophageal reflux disease (NERD) have no visible mucosal injury at the time of endoscopic examination, whereas others have esophagitis, peptic strictures, Barrett esophagus, or evidence of extraesophageal diseases such as chest pain, pulmonary symptoms, or ear, nose, and throat symptoms. GERD is a multifactorial process, one of the most common diseases, contributing to the expenditure in the United States of 4 to 5 billion dollars per year for antacid medications.

The prevalence of GERD differs, depending on whether the analysis is based on disease symptoms (e.g., heartburn) or signs (i.e., esophagitis). Based on symptoms, GERD is common in Western countries. The prevalence of heartburn and acid regurgitation in the past 12 months was noted to be 42% and 45%, respectively according to a study conduced by Locke and colleagues who mailed questionnaires to a predominantly white population residing in Olmsted County, Minn. (Locke G. R. et al. (1997) Gastroenterology 112: 1448). Frequent symptoms (at least weekly) were reported by 20% of respondents, with an equal gender distribution across all ages. The majority reported that heartburn was of moderate severity and had duration of 5 years or more, and only 5.4% reported a physician visit for reflux complaints within the previous year.

Increasing age is an important factor in the prevalence of complications of hyperacidic disorders, probably due to cumulative acid injury to the esophagus over time. In contrast, the prevalence of GERD and its complication is relatively low among residents of Africa and Asia. Possible reasons for the lower GERD prevalence includes low dietary fat, lower body mass index, and lower maximal acid output related to infection with Helicobacter pilori. However, the prevalence of GERD is increasing in Western countries. It has been reported that GERD is rarely a cause of death. Spechler S. J. (1992) Digestion 51 (Suppl. 1): 24. GERD, however, is associated with considerable morbidity and with complications such as esophageal ulcerations, peptic strictures and Barrett esophagus. Furthermore, GERD as a chronic disease significantly impairs quality of life. As compared with other chronic medical conditions, the impairment of quality of life resulting from GERD is similar to, or even greater than that resulting from arthritis, myocardial infarction, heart failure, or hypertension. The pathophysiology of GERD is complex and results from an imbalance between defensive factors protecting the esophagus, such as esophageal acid clearance, antireflux barriers and tissue resistance, and aggressive factors from the stomach content, such as gastric acidity and volume and duodenal contents. The intermittent nature of symptoms and esophagitis in many patients suggest that the aggressive and defensive factors are part of a delicately balanced system.

A variety of approaches have been employed in an attempt to design therapies to prevent hyperacid secretion. For example, antacids and alginates are still widely used. They have a short duration of action but are seen as inexpensive and safe. However, they do not provide a long term resolution of GERD. H₂ receptor antagonists, which inhibit the histamine receptor on the basolateral membrane of the parietal cell, have been widely prescribed for GERD. Their mode of action offers more potent and longer effect on gastric activity providing symptom relief and healing. Proton pump inhibitors, or PPIs, target against the H,K-ATPase. They are widely used, particularly in reflux esophagitis. Both of these treatments, H₂ receptor antagonists and PPIs, have greatly improved the quality of life for patients suffering from hyperacid secretion. However, there are an ever increasing number of patients that have experienced recurrent disease while still taking the drugs. Tytgat, G. N. J. (2004) Best Practice & Research Clinical Gastroentereology 18 (5): 67-72; Basu, K. K. et al. (2002) Eur. J. Gastroenterol. & Hepatol. 14: 1187-1192. For example, it has been estimated that about 30% of GERD patients remain symptomatic on standard dose of PPI. Lu, M. et al. (2007) Dig. Dis. Sci. 52: 2813-2820; Pfman, J. J. (2003) Am. J. Gastroenterol. 98(3), Suppl.; Becker, V. et al. (2007) Aliment Phramacol. Ther. 26: 1355-1360; Geibel, J. P. (2005) World J. Gastroenterol. 11(34): 5259-5265. Furthermore, PPIs have a short plasma half life which often leads to nocturnal acid breakthrough. Therapeutic oral doses of PPIs reach steady state and thus achieve their maximal effective levels only after 4-5 days with typical dosing regiments. This slow and cumulative onset of effect of PPI drug is due to their ability to inhibit only those pumps which are active when the PPIs are available. After PPI administration, there is a return of acid secretion that is partly due to de novo synthesis of the enzyme. Shin, J. M. et al. (2006) Dig. Dis. Sci. 51: 823-833; Munson, K. (2005) Biochem. 44(14); 5267-5284; Sachs, G. et al. (2007) J. Clin. Gastroenterol. 41, supp 2.

Despite their high degree of efficacy and worldwide clinical use, failure in the treatment of acid related diseases has been reported. Furthermore, the degree and speed of onset of symptom relief are very important to patients.

### SUMMARY OF THE INVENTION

The present invention provides methods for treating or preventing a hyperacidic disorder comprising administering an effective amount of a calcilytic compound or a pharmaceutically acceptable salt thereof to a subject in need thereof. In one aspect, the hyperacidic disorder is caused by a Helicobacter pylori colonization, hiatus hernia, gastritis, active duodenal ulcers, gastric ulcers, Zollinger-Ellison syndrome, dyspepsia, duodenogastric reflux, or delayed gastric emptying. The hyperacidic disorder can be GERD or NERD. In one aspect, GERD includes peptic esophageal strictures, Barrett esophagus, gastric adenocarcinoma. In a further aspect, GERD may be mild, moderate or severe.

The invention provides methods for treating or preventing of a hyperacidic disorder further comprising administering an effective amount of a compound for treating heartburn, a compound for treating acid regurgitation, a compound for treating dysphagia, a compound for treating water brash, odynophagia, burping, hiccups, nausea, or vomiting or a compound for treating non-cardiac chest pain, asthma, posterior laryngitis, reflux laryngitis, chronic cough, recurrent pneumonitis, or dental erosion.

In one aspect, the methods of the invention further comprise a lifestyle modification. The lifestyle modification may include the head of the bed elevation, avoidance of tight-fitting clothes, weight loss, restriction of alcohol, elimination of smoking, dietary therapy, refraining from lying down after meals, and avoidance of evening snacks before bedtime.

In one aspect, the methods of the invention further comprise administering an antacid. In another aspect, the methods of the invention further comprise administering a buffering agent. In a further aspect, the methods of the invention further comprise administering a prokinetic. In another aspect, the methods of the invention further comprise administering an H₂ receptor antagonist. In one aspect, the methods of the invention further comprise administering a proton pump inhibitor. In one aspect, the methods of the invention further comprise administering maintenance therapy. In another aspect, the methods of the invention further comprise administering a calcimimetic compound.

The invention provides methods for treatment of a hyperacidic disorder comprising administering an effective amount of a calcimimetic compound or a pharmaceutically acceptable salt thereof in combination with a PPI to a subject in need thereof.

In one aspect, the calcilytic compound is 2-chloro-6-(2-hydroxy-3-(2-methyl-1-(naphthalen-2-yl)propan-2-ylamino)propoxy)benzonitrile. Other calcilytic and calcimimetic compounds useful in the methods of the present invention are described in detail in Detailed Description below.

In one aspect, the subject can be mammal. In one aspect, the subject can be human. In a further aspect, the human subject can be elderly. In another aspect, the human subject can be pregnant.

### BRIEF DESCRIPTION OF THE DRAWINGS

FIG. 1 illustrates that the calcimimetic Compound A increases acid secretion by gastric parietal cells in the in vitro gland isolated from mice (upper panel) or rats (lower panel) that express the functional calcium sensing receptor. Acid induced by Compound A is compared to that induced by the cholinergic agonist, carbachol.

FIG. 2 demonstrates that Compound A is unable to increase acid secretion by superfused gastric glands in mice where the calcium sensing receptor gene is deleted (Casr^{-/-}; Gcm2^{-/-}). However, secretagogues like histamine or carbachol are able to increase acid secretion by gastric glands from these mice

FIG. 3 illustrates that the calcilytic compound B reduces acid secretion in a dose-dependent manner by superfused gastric glands isolated from mice that express the functional calcium-sensing receptor (Casr^{+/+}; Gcm2^{-/-}).

FIG. 4 schematically represents the effect of calcimimetics and calcilytics to modulate acid secretion by the gastric parietal cell. Calcimimetics activate the calcium sensing receptor and stimulate acid secretion by the gastric H,K-ATPase proton pump. In contrast, calcilytics inhibit the calcium sensing receptor and reduce acid secretion by the gastric H,K-ATPase proton pump even when the pump is mutated to make it constitutively active.

FIG. 5 illustrates the dose-dependent effect of calcilytic Compound B to reduce acid secretion by the superfused gastric gland isolated from mice that express the functional calcium sensing receptor and have a constitutively active gastric H,K-ATPase proton pump.

FIG. 6 demonstrates that when cells are activated first by a hormonal secretagogue, when, for example, this secretagogue released after a meal, the addition of a calcimimetic can inhibit acid secretion as demonstrated in superfused gastric glands isolated from Sprague-Dawley rats.

### DETAILED DESCRIPTION OF THE INVENTION

### I. Definitions

As used herein, the term “subject” is intended to mean a human, or an animal, in need of a treatment. This subject can have, or be at risk of developing, a bowel disorder, for example, inflammatory bowel disorder or irritable bowel syndrome.

“Treating” or “treatment” of a disease includes: (1) preventing the disease, i.e., causing the clinical symptoms of the disease not to develop in a subject that may be or has been exposed to the disease or conditions that may cause the disease, or predisposed to the disease but does not yet experience or display symptoms of the disease, (2) inhibiting the disease, i.e., arresting or reducing the development of the disease or any of its clinical symptoms, or (3) relieving the disease, i.e., causing regression of the disease or any of its clinical symptoms.

Administration “in combination with” or “together with” one or more further therapeutic agents includes simultaneous or concurrent administration and consecutive administration in any order.

The phrase “therapeutically effective amount” is the amount of the compound of the invention that will achieve the goal of improvement in disorder severity and the frequency of incidence. The improvement in disorder severity includes the reversal of the disease, as well as slowing down the progression of the disease.

As used herein, “calcium sensing receptor” or “CaSR” refers to the G-protein-coupled receptor responding to changes in extracellular calcium and/or magnesium levels. Activation of the CaSR produces rapid, transient increases in cytosolic calcium concentration by mobilizing calcium from thapsigargin-sensitive intracellular stores and by increasing calcium influx though voltage-insensitive calcium channels in the cell membrane (Brown et al., Nature 366: 575-580, 1993; Yamaguchi et al., Adv Pharmacol 47: 209-253, 2000). The phrase “hyperacidic disorders” includes, for example, gastroesophageal reflux disease, non-erosive reflux disease, duodenal ulcer disease, gastrointestinal ulcer disease, erosive esophagitis, poorly responsive symptomatic gastroesophageal reflux disease, pathological gastrointestinal hypersecretory disease, Zollinger Ellison Syndrome, acid dyspepsia, heartburn, chronic hyperacidic gastritis, and duodenogastric reflux. Each of these diseases is described in more detail in Methods of Treatment section below.

### II. Calcilytic and Calcimimetic Compounds and Pharmaceutical Compositions Comprising Them, Administration and Dosage

A. Calcilytic and Calcimimetic Compounds, Definitions

As used herein, the term “calcilytic compound” or “calcilytic” refers to compounds that inhibit, block, or decrease calcium sensing receptor (CaSR) activity, for examples, by causing a decrease in one or more calcium receptor activities evoked by extracellular Ca²⁺. In one aspect, calcilytic may block, either partially or completely, the ability of increased concentrations of extracellular Ca²⁺ to (a) increase [Ca²⁺ᵢ]; (b) mobilize intracellular Ca²⁺; (c) increase the formation of inositol-1,4,5-triphosphate; and (d) decrease dopamine or isoproterenol-stimulated cyclic AMP formation. In one aspect, a calcilytic compound can be a small molecule. In another aspect, a calcilytic can be an antagonistic antibody.

Calcilytic compounds useful in the present invention include those disclosed in, for example, European Patent and Publications Nos 637,237, 724,561, 901,459, 973,730, 1,258,471, 1,466,888, 1,509,518; International Publication Nos. WO 97/37967, WO 99/51569, WO 01/08673, WO 04/017908, WO 04/041755, WO 04/047751, WO 05/030746, WO 05/030749; WO05077886, WO05077892, W005108376, WO06041968, WO06042007, WO06066070 WO07062370, WO07044796, U.S. Pat. Nos. 6,395,919, 6,432,656, 6,521,667, 6,750,255, 6,818,660, 6,864,267, 6,908,935, 6,916,956, 6,939,895; 7,084,167; 7,109,238; 7,157,498; 7,202,261; 7,205,322; 7,211,685; 7,265,145, and U.S. Patent Application Publication Nos. 2002/0099220, 2004/0009980, 2004/0014723, 2004/0192741, and 2005/0032850.

As used herein, the term “calcimimetic compound” or “calcimimetic” refers to a compound that binds to calcium sensing receptors and induces a conformational change that reduces the threshold for calcium sensing receptor activation by the endogenous ligand Ca²⁺. These calcimimetic compounds can also be considered allosteric modulators of the calcium receptors.

In one aspect, a calcimimetic can have one or more of the following activities: it evokes a transient increase in internal calcium, having a duration of less that 30 seconds (for example, by mobilizing internal calcium); it evokes a rapid increase in [Ca²⁺ᵢ], occurring within thirty seconds; it evokes a sustained increase (greater than thirty seconds) in [Ca²⁺ᵢ] (for example, by causing an influx of external calcium); evokes an increase in inositol-1,4,5-triphosphate or diacylglycerol levels, usually within less than 60 seconds; and inhibits dopamine- or isoproterenol-stimulated cyclic AMP formation. In one aspect, the transient increase in [Ca²⁺ᵢ] can be abolished by pretreatment of the cell for ten minutes with 10 mM sodium fluoride or with an inhibitor of phospholipase C, or the transient increase is diminished by brief pretreatment (not more than ten minutes) of the cell with an activator of protein kinase C, for example, phorbol myristate acetate (PMA), mezerein or (−) indolactam V. In one aspect, a calcimimetic compound can be a small molecule. In another aspect, a calcimimetic can be an agonistic antibody to the CaSR.

Calcimimetic compounds useful in the present invention include those disclosed in, for example, European Patent No. 637,237, 657,029, 724,561, 787,122, 907,631, 933,354, 1,203,761, 1,235 797, 1,258,471, 1,275,635, 1,281,702, 1,284,963, 1,296,142, 1,308,436, 1,509,497, 1,509,518, 1,553,078; International Publication Nos. WO 93/04373, WO 94/18959, WO 95/11221, WO 96/12697, WO 97/41090, WO 01/34562, WO 01/90069, WO 02/14259, WO 02/059102, WO 03/099776, WO 03/099814, WO 04/017908; WO 04/094362, WO 04/106280, WO05115975; WO 06/117211; WO 06/123725; WO07060026; WO08006625; U.S. Pat. Nos. 5,688,938, 5,763,569, 5,962,314, 5,981,599, 6,001,884, 6,011,068, 6,031,003, 6,172,091, 6,211,244, 6,313,146, 6,342,532, 6,362,231, 6,432,656, 6,710,088, 6,750,255, 6,908,935, 7,084,167; 7,157,498, 7,176,322; 7,196,102, and U.S. Patent Application Publication No. 2002/0107406, 2003/0008876, 2003/0144526, 2003/0176485, 2003/0199497, 2004/0006130, 2004/0077619, 2005/0032796, 2005/0107448, 2005/0143426, 2007/0225296; European patent application PCT/EP2006/004166, EP 1882684.

In certain embodiments, the calcimimetic compound is chosen from compounds of Formula I and pharmaceutically acceptable salts thereof:

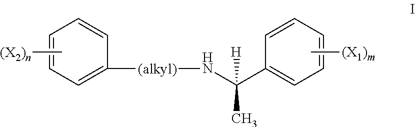

wherein:

X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;

n ranges from 0 to 5;

m ranges from 1 to 5; and

the alkyl radical is chosen from C1-C3 alkyl radicals, which are optionally substituted with at least one group chosen from saturated and unsaturated, linear, branched, and cyclic C1-C9 alkyl groups, dihydroindolyl and thiodihydroindolyl groups, and 2-, 3-, and 4-piperid(in)yl groups.

The calcimimetic compound may also be chosen from compounds of Formula II:

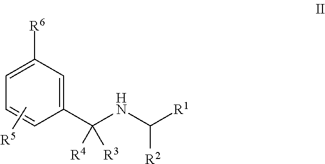

and pharmaceutically acceptable salts thereof,
wherein:

R¹ is aryl, substituted aryl, heterocyclyl, substituted heterocyclyl, cycloalkyl, or substituted cycloalkyl;

R² is alkyl or haloalkyl;

R³ is H, alkyl, or haloalkyl;

R⁴ is H, alkyl, or haloalkyl;

each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};

R⁶ is aryl, substituted aryl, heterocyclyl, substituted heterocyclyl, cycloalkyl, or substituted cycloalkyl;

each R^{a} is, independently, H, alkyl or haloalkyl;

each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;

each R^{c} is, independently, alkyl, haloalkyl, phenyl or benzyl, each of which may be substituted or unsubstituted;

each R^{d} is, independently, H, alkyl, aryl, aralkyl, heterocyclyl, or heterocyclylalkyl wherein the alkyl , aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, —C(50 O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a};

m is 1 or 2;

n is 0, 1 or 2; and

p is 0, 1, 2, 3, or 4;

provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl. These compounds are described in detail in published US patent application number 20040082625.

In one aspect, the calcimimetic compound can be N-((6-(methyloxy)-4′-(trifluoromethyl)-1,1′-biphenyl-3-yl)methyl)-1-phenylethanamine, or a pharmaceutically acceptable salt thereof. In another aspect, the calcimimetic compound can be (1R)-N-((6-chloro-3′-fluoro-3-biphenylypmethyl)-1-(3-chlorophenyl)ethanamine, or a pharmaceutically acceptable salt thereof. In a further aspect, the calcimimetic compound can be (1R)-1-(6-(methyloxy)-4′-(trifluoromethyl)-3-biphenylyl)-N-((1R)-1-phenylethyl)ethanamine, or a pharmaceutically acceptable salt thereof.

In certain embodiments of the invention the calcimimetic compound can be chosen from compounds of Formula III

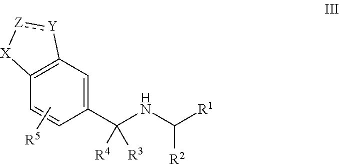

and pharmaceutically acceptable salts thereof, wherein:

[symbol] represents a double or single bond;

R¹ is R^{b};

R² is C₁₋₈ alkyl or C₁₋₄ haloalkyl;

R³ is H, C₁₋₄ haloalkyl or C₁₋₈ alkyl;

R⁴ is H, C₁₋₄ haloalkyl or C₁₋₄ alkyl;

R⁵ is, independently, in each instance, H, C₁₋₈alkyl, C₁₋₄haloalkyl, halogen, —OC₁₋₆alkyl, —NR^{a}n^{d} or NR^{d}C(═O)R^{d};

X is —CR^{d}═N—, —N═CR^{d}—, O, S or —NR^{d}—;

when [symbol] is a double bond then Y is ═CR⁶— or ═N— and Z is —CR⁷═ or —N═; and when [symbol] is a single bond then Y is —CR^{a}R⁶— or —NR^{d}— and Z is —CR^{a}R⁷— or —NR^{d}—; and

R⁶ is R^{d}, C₁₋₄haloalkyl, —C(═O)R^{c}, —OC₁₋₆alkyl, —OR^{b}, —NR^{a}_{R}^{a}, —NR^{a}R^{b}, —C(═O)OR^{c}, —C(═O)NR^{a}R^{a}, —OC(═O)R^{c}, —NR^{a}C(═O)R^{c}, cyano, nitro, -NR^{a}S(═O)ₘR^{c} or —S(═O)ₘNR^{a}R^{a};

R⁷ is R^{d}, C₁₋₄haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, -NR^{a}S(═O)ₘR^{c} or —S(═O)ₘNR^{a}R^{a}; or R⁶ and R⁷ together form a 3- to 6-atom saturated or unsaturated bridge containing 0, 1, 2 or 3 N atoms and 0, 1 or 2 atoms selected from S and O, wherein the bridge is substituted by 0, 1 or 2 substituents selected from R⁵; wherein when R⁶ and R⁷ form a benzo bridge, then the benzo bridge may be additionally substituted by a 3- or 4-atoms bridge containing 1 or 2 atoms selected from N and O, wherein the bridge is substituted by 0 or 1 substituents selected from C₁₋₄alkyl;

R^{a} is, independently, at each instance, H, C₁₋₄haloalkyl or C₁₋₆alkyl;

R^{b} is, independently, at each instance, phenyl, benzyl, naphthyl or a saturated or unsaturated 5- or 6-membered ring heterocycle containing 1, 2 or 3 atoms selected from N, O and S, with no more than 2 of the atoms selected from O and S, wherein the phenyl, benzyl or heterocycle are substituted by 0, 1, 2 or 3 substituents selected from C₁₋₆alkyl, halogen, C₁₋₄haloalkyl, —OC₁₋₆alkyl, cyano and nitro;

R^{c} is, independently, at each instance, C₁₋₆alkyl, C₁₋₄haloalkyl, phenyl or benzyl;

R^{d} is, independently, at each instance, H, C₁₋₆alkyl, phenyl, benzyl or a saturated or unsaturated 5- or 6-membered ring heterocycle containing 1, 2 or 3 atoms selected from N, O and S, with no more than 2 of the atoms selected from O and S, wherein the C₁₋₆ alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₘR^{c} and —S(═O)ₘNR^{a}R^{a}; and

m is 1 or 2.

Compounds of Formula III are described in detail in U.S. patent application 20040077619.

In one aspect, a calcimimetic compound is N-(3-[2-chlorophenyyl]-propyl)-R-▪-methyl-3-methoxybenzylamine HCl (Compound A). In another aspect, a calcimimetic compound is N-((6-(methyloxy)-4′-(trifluoromethyl)-1,1′-biphenyl-3-yl)methyl)-1-phenylethanamine

In one aspect, the calcimimetic compound of the invention can be chosen from compounds of Formula IV

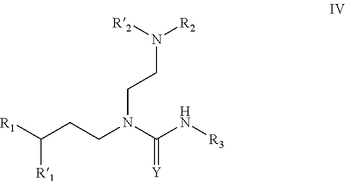

wherein:

Y is oxygen or sulphur;

R₁ and R′₁ are the same or different, and each represents an aryl group, a heteroaryl group, or R₁ and R′₁, together with the carbon atom to which they are linked, form a fused ring structure of formula:

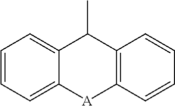

in which A represents a single bond, a methylene group, a dimethylene group, oxygen,

nitrogen or sulphur, said sulphur optionally being in the sulphoxide or sulphone forms, wherein each of R₁ and R′₁, or said fused ring structure formed thereby, is optionally substituted by at least one substituent selected from the group c,

wherein the group c consists of: halogen atoms, hydroxyl, carboxyl, linear and branched alkyl, hydroxyalkyl, haloalkyl, alkylthio, alkenyl, and alkynyl groups; linear and branched alkoxyl groups; linear and branched thioalkyl groups; hydroxycarbonylalkyl;

alkylcarbonyl; alkoxycarbonylalkyl; alkoxycarbonyl; trifluoromethyl; trifluoromethoxyl; —CN; —NO₂; alkylsulphonyl groups optionally in the sulphoxide or sulphone forms; wherein any alkyl component has from 1 to 6 carbon atoms, and any alkenyl or alkynyl components have from 2 to 6 carbon atoms,

and wherein, when there is more than one substituent, then each said substituent is the same or different,

R₂ and R′₂, which may be the same or different, each represents: a hydrogen atom; a linear or branched alkyl group containing from 1 to 6 carbon atoms and optionally substituted by at least one halogen atom, hydroxy or alkoxy group containing from 1 to 6 carbon atoms; an alkylaminoalkyl or dialkylaminoalkyl group wherein each alkyl group contains from 1 to 6 carbon atoms,

or R₂ and R′₂, together with the nitrogen atom to which they are linked, form a saturated or unsaturated heterocycle containing 0, 1 or 2 additional heteroatoms and having 5, 6, or 7 ring atoms, said heterocycle being optionally substituted by at least one substituent selected from the group ‘c’ defined above,

and wherein, when there is more than one substituent, said substituent is the same or different,

R₃ represents a group of formula:

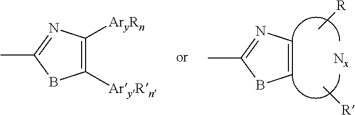

in which B represents an oxygen atom or a sulphur atom, x is 0, 1 or 2, y and y′ are the same or different, and each is 0 or 1, Ar and Ar′ are the same or different and each represents an aryl or heteroaryl group, n and n′ are the same or different, and each is 1, when the y or y′ with which it is associated is 0, or is equal to the number of positions that can be substituted on the associated Ar or Ar′ when the said y or y′ is 1, the fused ring containing Nₓ is a five- or six-membered heteroaryl ring, and wherein R and R′, which may be the same or different, each represent a hydrogen atom or a substituent selected from the group a,

wherein the group a consists of: halogen atoms; hydroxyl; carboxyl; aldehyde groups; linear and branched alkyl, alkenyl, alkynyl, hydroxyalkyl, hydroxyalkenyl, hydroxyalkynyl, haloalkyl, haloalkenyl, and haloalkynyl groups; linear and branched alkoxyl groups; linear and branched thioalkyl groups; aralkoxy groups; aryloxy groups; alkoxycarbonyl; aralkoxycarbonyl; aryloxycarbonyl; hydroxycarbonylalkyl; alkoxycarbonylalkyl; aralkoxycarbonylalkyl; aryloxycarbonylalkyl; perfluoroalkyl; perfluoroalkoxy; —CN; acyl; amino, alkylamino, aralkylamino, arylamino, dialkylamino, diaralkylamino, diarylamino, acylamino, and diacylamino groups; alkoxycarbonylamino, aralkoxycarbonylamino, aryloxycarbonylamino, alkylcarbonylamino, aralkylcarbonylamino, and arylcarbonylamino groups; alkylaminocarbonyloxy, aralkylaminocarbonyloxy, and arylaminocarbonyloxy groups; alkyl groups substituted with an amino, alkylamino, aralkylamino, arylamino, dialkylamino, diaralkylamino, diarylamino, acylamino, trifluoromethylcarbonyl-amino, fluoroalkylcarbonylamino, or diacylamino group; CONH₂; alkyl-, aralkyl-, and aryl-amido groups; alkylthio, arylthio and aralkylthio and the oxidised sulphoxide and sulphone forms thereof; sulphonyl, alkylsulphonyl, haloalkylsulphonyl, arylsulphonyl and aralkylsulphonyl groups; sulphonamide, alkylsulphonamide, haloalkylsulphonamide, di(alkylsulphonyl)amino, aralkylsulphonamide, di(aralkylsulphonyl)amino, arylsulphonamide, and di(arylsulphonyl)amino; and saturated and unsaturated heterocyclyl groups, said heterocyclyl groups being mono- or bicyclic and being optionally substituted by one or more substituents, which may be the same or different, selected from the group b,

wherein the group b consists of: halogen atoms; hydroxyl; carboxyl; aldehyde groups; linear and branched alkyl, alkenyl, alkynyl, hydroxyalkyl, hydroxyalkenyl, hydroxyalkynyl, haloalkyl, haloalkenyl, and haloalkynyl groups; linear and branched alkoxyl groups; linear and branched thioalkyl groups; alkoxycarbonyl; hydroxycarbonylalkyl; alkoxycarbonylalkyl; perfluoroalkyl; perfluoroalkoxy; —CN; acyl; amino, alkylamino, dialkylamino, acylamino, and diacylamino groups; alkyl groups substituted with an amino, alkylamino, dialkylamino, acylamino, or diacylamino group; CONH₂; alkylamido groups; alkylthio and the oxidised sulphoxide and sulphone forms thereof sulphonyl, alkylsulphonyl groups; and sulphonamide, alkylsulphonamide, and di(alkylsulphonyl)amino groups,

wherein, in groups a and b, any alkyl components contain from 1 to 6 carbon atoms, and any alkenyl or alkynyl components contain from 2 to 6 carbon atoms, and are optionally substituted by at least one halogen atom or hydroxy group, and wherein any aryl component is optionally a heteroaryl group.

In one aspect, the calcimimetic compound can be 3-(1,3-benzothiazol-2-yl)-1-(3,3-diphenylpropyl)-1-(2-(4-morpholinyl)ethyl)urea or pharmaceutically acceptable salt thereof. In another aspect, the calcimimetic compound can be N-(4-(2-((((3,3-diphenylpropyl)(2-(4-morpholinyl)ethyl)amino)carbonyl)amino)-1,3-thiazol-4-yl)phenyl)methanesulfonamide or pharmaceutically acceptable salt thereof.

In one aspect, the calcimimetic compound of the invention can be chosen from compounds of Formula V

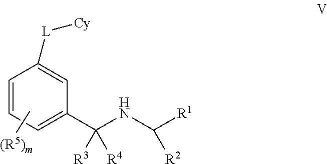

wherein:

R¹ is phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro;

R² is C₁₋₈alkyl or C₁₋₄haloalkyl;

R³ is H, C₁₋₄haloalkyl or C₁₋₈alkyl;

R⁴ is H, C₁₋₄haloalkyl or C₁₋₈alkyl;

R⁵ is, independently, in each instance, H, C₁₋₈alkyl, C₁₋₄haloalkyl, halogen, —OC₁₋₆alkyl, —NR^{a}n^{d}, NR^{a}C(═O)R^{d}, substituted or unsubstituted pyrrolidinyl, substituted or unsubstituted azetidinyl, or substituted or unsubstituted piperidyl, wherein the substituents can be selected from halogen, —OR^{b}, —NR^{a}R^{d}, —C(═O)OR^{c}, —C(═O)NR^{a}R^{d}, —OC(═O)R^{c}, —NR^{a}C(═O)R^{c}, cyano, nitro, —NR^{a}S(═O)ₙR^{c} or —S(═O)ₙNR^{a}n^{d};

L is —O—, —OC₁₋₆alkyl-, -C₁₋₆alkylO—, —N(R^{a})(R^{d})—, —NR^{a}C(═O)—, —C(═O)NR^{d}C₁₋₆alkyl-, -C₁₋₆alkyl-C(═O)NR^{d}—, —NR^{d}C(═O)NR^{d}—, —NR^{d}C(═O)NR^{d}C₁₋₆alkyl-, —NR^{a}C(═O)R^{c}—, —NR^{a}C(═O)OR^{c}—, —OC₁₋₆alkyl-C(═O)O—, —NR^{d}C₁₋₆alkyl-, -C₁₋₆alkylNR^{d}—, —S—, —S(═O)ₙ—, —NR^{a}S(═O)ₙ, or —S(═O)ₙN(R^{a})—;

Cy is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of R⁶, C₁₋₈alkyl, C₁₋₄haloalkyl, halogen, cyano, nitro, —OC₁₋₆alkyl, —NR^{a}R^{d}, NR^{d}C(═O)R^{d}, —C(═O)R^{c}, —C(═O)NR^{a}R^{d}, —OC(═O)R^{c}, —NR^{a}C(═O)R^{c}, —NR^{a}S(═O)ₙR^{c} or —S(═O)ₘNR^{a}n^{d};

R⁶ is a partially or fully saturated or unsaturated 5-8 membered monocyclic, 6-12 membered bicyclic, or 7-14 membered tricyclic ring system, the ring system formed of carbon atoms optionally including 1-3 heteroatoms if monocyclic, 1-6 heteroatoms if bicyclic, or 1-9 heteroatoms if tricyclic, and wherein each ring of the ring system is optionally substituted independently with one or more substituents of C₁₋₈alkyl, C₁₋₄haloalkyl, halogen, cyano, nitro, —OC₁₋₆alkyl, —NR^{a}R^{d}, NR^{d}C(═O)R^{d} , —C(═O)OR^{c}, —C(═O)NR^{a}R^{d}, —OC(═O)R^{c}, —NR^{a}C(═O)R^{c}, —NR^{a}S(═O)ₘR^{c} or —S(═O)ₘNR^{a}R^{d};

R^{a} is, independently, at each instance, H, C₁₋₄haloalkyl, C₁₋₆alkyl, C₁₋₆alkenyl, C₁₋₆alkylaryl or arylC₁₋₆alkyl;

R^{b} is, independently, at each instance, C₁₋₈alkyl, C₁₋₄haloalkyl, phenyl, benzyl, naphthyl or a saturated or unsaturated 5- or 6-membered heterocyclic ring containing 1, 2 or 3 atoms selected from N, O and S, with no more than 2 of the atoms selected from O and S, wherein the phenyl, benzyl, naphthyl or heterocyclic ring are substituted by 0, 1, 2 or 3 substituents selected from C₁₋₆alkyl, halogen, C₁₋₄haloalkyl, —OC₁₋₆alkyl, cyano and nitro;

R^{c} is, independently, at each instance, C₁₋₆alkyl, C₁₋₄haloalkyl, phenyl or benzyl;

R^{d} is, independently, at each instance, H, C₁₋₆alkyl, C₁₋₆alkenyl, phenyl, benzyl, naphthyl or a saturated or unsaturated 5- or 6-membered heterocycle ring containing 1, 2 or 3 atoms selected from N, O and S, with no more than 2 of the atoms selected from O and S, wherein the C₁₋₆alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, —NR^{a}R^{b}, —C(═O)OR^{c}, —C(═O)NR^{a}R^{b}, —OC(═O)R^{c}, —NR^{a}C(═O)R^{c}, —NR^{a}S(═O)ₘR^{c} and —S(═O)ₘNR^{a}R^{a};

m is 1 or 2;

n is 1 or 2;

provided that if L is —O— or —OC₁₋₆alkyl-, then Cy is not phenyl.

In one aspect, the calcimimetic compound can be N-(2-chloro-5-((((1R)-1-phenylethyl)amino)methyl)phenyl)-5-methyl-3-isoxazolecarboxamide or a pharmaceutically acceptable salt thereof. In another aspect, the calcimimetic compound can be N-(2-chloro-5-((((1R)-1-phenylethyl)amino)methyl)phenyl)-2-pyridinecarboxamide or a pharmaceutically acceptable salt thereof.

Calcimimetic compounds useful in the methods of the invention include the calcimimetic compounds described above, as well as their stereoisomers, enantiomers, polymorphs, hydrates, and pharmaceutically acceptable salts of any of the foregoing.

Calcilytic and calcimimetic compounds useful in the methods of the invention include the calcilytic and calcimimetic compounds described above, as well as their stereoisomers, enantiomers, polymorphs, hydrates, and pharmaceutically acceptable salts of any of the foregoing. Further, compounds identified as calcilytic and calcimimetic by methods described below can be used in the methods of the present invention.

B. Methods of Assessing Calcilytic Activity

In one aspect, compounds binding at the CaSR-activity modulating site can be identified using, for example, a labeled compound binding to the site in a competition-binding assay format.

Calcilytic activity of a compound can be determined using techniques such as those described in International Publications WO 93/04373, WO 94/18959 and WO 95/11211.

Other methods that can be used to assess compounds calcilytic activity are described below.

Calcium Receptor Inhibitor Assay

Calcilytic activity can be measured by determining the IC₅₀ of the test compound for blocking increases of intracellular Ca²⁺ elicited by extracellular Ca²⁺ in HEK 293 4.0 7 cells stably expressing the human calcium receptor. HEK 293 4.0 7 cells are constructed as described by Rogers et al., J. Bone Miner. Res. 10 Suppl. 1:S483, 1995. Intracellular Ca²⁺ increases were elicited by increasing extracellular Ca²⁺ from 1 to 1.75 mM. Intracellular Ca²⁺ was measured using fluo-3, a fluorescent calcium indicator.

Cells are maintained in T-150 flasks in selection media (DMEM supplemented with 10% fetal bovine serum and 200 μg/mL hygromycin B), under 5% CO₂:95% air at 37° C. and grown to 90% confluency.

The medium is decanted and the cell monolayer is washed twice with phosphate-buffered saline (PBS) kept at 37° C. After the second wash, 6 mL of 0.02% EDTA in PBS is added and incubated for 4 minutes at 37° C. Following the incubation, cells are dispersed by gentle agitation. Cells from 2 or 3 flasks are pooled and pelleted (100×g). The cellular pellet is resuspended in 15 mL of SPF-PCB+ and pelleted again by centrifugation. This washing is done twice. Sulfate- and phosphate-free parathyroid cell buffer (SPF-PCB) contains 20 mM Na-Hepes, pH 7.4, 126 mM NaCl, 5 mM KCl, and 1 mM MgCl₂. SPF-PCB is made up and stored at 4° C. On the day of use, SPF-PCB is supplemented with 1 mg/mL of D-glucose and 1 mM CaCl₂ and then split into two fractions. To one fraction, bovine serum albumin (BSA; fraction V, ICN) is added at 5 mg/mL (SPF-PCB+). This buffer is used for washing, loading and maintaining the cells. The BSA-free fraction is used for diluting the cells in the cuvette for measurements of fluorescence. The pellet is resuspended in 10 mL of SPF-PCB+ containing 2.2 μM fluo-3 (Molecular Probes) and incubated at room temperature for 35 minutes. Following the incubation period, the cells are pelleted by centrifugation. The resulting pellet is washed with SPF-PCB+. After washing, cells are resuspended in SPF-PCB+ at a density of 1 2×10⁶ cells/mL. For recording fluorescent signals, 300 μL of cell suspension are diluted in 1.2 mL of SPF buffer containing 1 mM CaCl₂ and 1 mg/mL of D-glucose. Fluorescence measurements are performed at 37° C. with constant stirring using a spectrofluorimeter. Excitation and emission wavelengths are measured at 485 and 535 nm, respectively. To calibrate fluorescence signals, digitonin (5 mg/mL in ethanol) is added to obtain Fmax, and the apparent Fmin is determined by adding Tris-EGTA (2.5 M Tris-Base, 0.3 M EGTA). The concentration of intracellular calcium is calculated using the following equation: intracellular calcium=(F-Fₘᵢₙ/Fₘₐₓ)×K_{d}; where K_{d}=400 nM. To determine the potential calcilytic activity of test compounds, cells are incubated with test compound (or vehicle as a control) for 90 seconds before increasing the concentration of extracellular Ca²⁺ from 1 to 2 mM. Calcilytic compounds are detected by their ability to block, in a concentration-dependent manner, increases in the concentration of intracellular Ca²⁺ elicited by extracellular Ca²⁺.

In general, compounds having lower IC₅₀ values in the Calcium Receptor Inhibitor Assay, for example, IC₅₀ of 1 uM or lower are useful in the methods of the instant invention.

Calcium Receptor Binding Assay

HEK 293 4.0 7 cells stably transfected with the Human Calcium Receptor are grown in T180 tissue culture flasks. Plasma membrane is obtained by polytron homogenization or glass douncing in buffer (50 mM Tris-HCl pH 7.4, 1 mM EDTA, 3 mM MgCl₂) in the presence of a protease inhibitor cocktail containing 1 μM Leupeptin, 0.04 μM Pepstatin, and 1 mM PMSF. Aliquoted membrane was snap frozen and stored at −80° C. ³H-labeled compound is radiolabeled to a radiospecific activity of 44 Ci/mmole and is aliquoted and stored in liquid nitrogen for radiochemical stability.

A typical reaction mixture contains 2 nM ³H-labeled compound ((R,R)-N-4′-Methoxy-t-3-3′-methyl-1′-ethylphenyl-1-(1-naphthyl)ethylamine-), or ³H-labeled compound (R)-N-[2-Hydroxy-3-(3-chloro-2-cyanophenoxy)propyl]-1,1-dimethyl-2-(4-methoxyphenyl)ethylamine 4 10 μg membrane in homogenization buffer containing 0.1% gelatin and 10% EtOH in a reaction volume of 0.5 mL. Incubation is performed in 12×75 polyethylene tubes in an ice water bath. To each tube 25 μL of test sample in 100% EtOH is added, followed by 400 μL of cold incubation buffer, and 25 μL of 40 nM ³H-compound in 100% EtOH for a final concentration of 2 nM. The binding reaction is initiated by the addition of 50 μL of 80 200 μg/mL HEK 293 4.0 7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting.

C. Methods of Assessing Calcimimetic Activity

HEK 293 Cell Assay

HEK 293 cells engineered to express human CaSR (HEK 293 4.0-7) have been described in detail previously (Nemeth E F et al. (1998) Proc. Natl. Acad. Sci. USA 95:4040-4045). This clonal cell line has been used extensively to screen for agonists, allosteric modulators, and antagonists of the CaSR (Nemeth E F et al. (2001) J. Pharmacol. Exp. Ther. 299:323-331).

For measurements of cytoplasmic calcium concentration, cells are recovered from tissue culture flasks by brief treatment with 0.02% ethylenediaminetetraacetic acid (EDTA) in phosphate-buffered saline (PBS) and washed and resuspended in Buffer A (126 mM NaCl, 4 mM KCl, 1 mM CaCl₂, 1 mM MgSO₄, 0.7 mM K₂HPO₄/KH₂PO₄, 20 mM Na-Hepes, pH 7.4) supplemented with 0.1% bovine serum albumin (BSA) and 1 mg/ml D-glucose. The cells are loaded with fura-2 by incubation for 30 minutes at 37° C. in Buffer A and 2 μM fura-2 acetoxymethylester. The cells are washed with Buffer B (Buffer B is Buffer A lacking sulfate and phosphate and containing 5 mM KCl, 1 mM MgCl₂, 0.5 mM CaCl₂ supplemented with 0.5% BSA and 1 mg/ml D-glucose) and resuspended to a density of 4 to 5×10⁶ cells/ml at room temperature. For recording fluorescent signals, the cells are diluted five-fold into prewarmed (37° C.) Buffer B with constant stirring. Excitation and emission wavelengths are 340 and 510 nm, respectively. The fluorescent signal is recorded in real time using a strip-chart recorder.

For fluorometric imaging plate reader (FLIPR) analysis, HEK 293 cells are maintained in Dulbecco's modified Eagle's medium (DMEM) with 10% fetal bovine serum (FBS) and 200 μg/ml hygyomycin. At 24 hrs prior to analysis, the cells are trypsinized and plated in the above medium at 1.2×10⁵ cells/well in black sided, clear-bottom, collagen 1-coated, 96-well plates. The plates are centrifuged at 1,000 rpm for 2 minutes and incubated under 5% CO₂ at 37° C. overnight. Cells are then loaded with 6 μM fluo-3 acetoxymethylester for 60 minutes at room temperature. All assays are performed in a buffer containing 126 mM NaCl, 5 mM KCl, 1 mM MgCl₂, 20 mM Na-Hepes, supplemented with 1.0 mg/ml D-glucose and 1.0 mg/ml BSA fraction IV (pH 7.4).

In one aspect, the EC₅₀'s for CaSR-active compounds can be determined in the presence of 1 mM Ca²⁺. The EC₅₀ for cytoplasmic calcium concentration can be determined starting at an extracellular Ca²⁺ level of 0.5 mM. FLIPR experiments are done using a laser setting of 0.8 W and a 0.4 second CCD camera shutter speed. Cells are challenged with calcium, CaSR-active compound or vehicle (20 μl) and fluorescence monitored at 1 second intervals for 50 seconds. Then a second challenge (50 μl) of calcium, CaSR-active compound, or vehicle can be made and the fluorescent signal monitored. Fluorescent signals are measured as the peak height of the response within the sample period. Each response is normalized to the maximum peak observed in the plate to determine a percentage maximum fluorescence.

Bovine Parathyroid Cells

The effect of calcimimetic compounds on CaSR-dependent regulation of PTH secretion can be assessed using primary cultures of dissociated bovine parathyroid cells. Dissociated cells can be obtained by collagenase digestion, pooled, then suspended in Percoll purification buffer and purified by centrifugation at 14,500×g for 20 minutes at 4° C. The dissociated parathyroid cells are removed and washed in a 1:1 mixture of Ham's F-12 and DMEM (F-12/DMEM) supplemented with 0.5% BSA, 100 U/ml penicillin, 100 μg/ml streptomycin, and 20 μg/ml gentamicin. The cells are finally resuspended in F-12/DMEM containing 10 U/ml penicillin, 10 μg/ml streptomycin, and 4 μg/ml gentamicin, and BSA was substituted with ITS+ (insulin, transferrin, selenous acid, BSA, and linoleic acid; Collaborative Research, Bedford, Mass.). Cells are plated into T-75 flasks and grown at 37° C. in a humidified atmosphere of 5% CO₂ in air.

Following overnight culture, the cells are removed from flasks by decanting and washed with parathyroid cell buffer (126 mM NaCl, 4 mM KCl, 1 mM MgSO₄, 0.7 mM K₂HPO₄/KH₂PO₄, 20 mM Na-Hepes, 20; pH 7.45 and variable amounts of CaCl₂ as specified) containing 0.1% BSA and 0.5 mM CaCl₂. The cells are resuspended in this same buffer and portions (0.3 ml) are added to polystyrene tubes containing appropriate controls, CaSR-active compound, and/or varying concentrations of CaCl₂. Each experimental condition is performed in triplicate. Incubations at 37° C. are for 20 minutes and can be terminated by placing the tubes on ice. Cells are pelleted by centrifugation (1500×g for 5 minutes at 4° C.) and 0.1 ml of supernatant is assayed immediately. A portion of the cells is left on ice during the incubation period and then processed in parallel with other samples. The amount of PTH in the supernatant from tubes maintained on ice is defined as “basal release” and subtracted from other samples. PTH is measured according to the vendor's instructions using rat PTH immunoradiometric assay kit (Immunotopics, San Clemente, Calif.).

MTC 6-23 Cell Calcitonin Release

Rat MTC 6-23 cells (clone 6), purchased from ATCC (Manassas, Va.) are maintained in growth media (DMEM high glucose with calcium/15% HIHS) that is replaced every 3 to 4 days. The cultures are passaged weekly at a 1:4 split ratio. Calcium concentration in the formulated growth media is calculated to be 3.2 mM. Cells are incubated in an atmosphere of 90% O₂/10% CO₂, at 37° C. Prior to the experiment, medium from sub-confluent cultures is aspirated and the cells rinsed once with trypsin solution. The trypis rinse is removed and fresh trypsin solution is added and incubated at room temperature for 5-10 minutes to detach the cells. Detached cells are suspended at a density of 3.0×10⁵ cells/mL in growth media and seeded at a density of 1.5×10⁵ cells/well (0.5 mL cell suspension) in collagen-coated 48 well plates (Becton Dickinson Labware, Bedford, Mass.). The cells are allowed to adhere for 56 hours post-seeding, after which growth media is aspirated and replaced with 0.5 mL of assay media (DMEM high glucose without/2% FBS). The cells are then incubated for 16 hours prior to determination of calcium-stimulated calcitonin release. The actual calcium concentration in this media is calculated to be less than 0.07 mM. To measure calcitonin release, 0.35 mL of test agent in assay media is added to each well and incubated for 4 hours prior to determination of calcitonin content in the media. Calcitonin levels are quantified according to the vendor's instructions using a rat calcitonin immunoradiometric assay kit (Immutopics, San Clemente, Calif.).

Inositol Phosphate Assay

The calcimimetic properties of compounds could also be evaluated in a biochemical assay performed on Chinese hamster ovarian (CHO) cells transfected with an expression vector containing cloned CaSR from rat brain [CHO(CaSR)] or not [CHO(WT)] (Ruat M., Snowman A M., J. Biol. Chem 271, 1996, p 5972). CHO (CaSR) has been shown to stimulate tritiated inositol phosphate ([³H]IP) accumulation upon activation of the CaSR by Ca²⁺ and other divalent cations and by R-568 (Ruat et al., J. Biol. Chem 271, 1996). Thus, [³H]IP accumulation produced by 10 μM of each CaSR-active compound in the presence of 2 mM extracellular calcium can be measured and compared to the effect produced by 10 mM extracellular calcium, a concentration eliciting maximal CaSR activation (Dauban P. et al., Bioorganic & Medicinal Chemistry Letters, 10, 2000, p 2001).

D. Pharmaceutical Compositions and Administration

Calcilytic compounds useful in the present invention can be used in the form of pharmaceutically acceptable salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mandelate, methansulfonate, nicotinate, 2-naphthalenesulfonate, oxalate, palmoate, pectinate, persulfate, 2-phenylpropionate, picrate, pivalate, propionate, salicylate, succinate, sulfate, tartrate, thiocyanate, tosylate, mesylate, and undecanoate. When compounds of the invention include an acidic function such as a carboxy group, then suitable pharmaceutically acceptable salts for the carboxy group are well known to those skilled in the art and include, for example, alkaline, alkaline earth, ammonium, quaternary ammonium cations and the like. For additional examples of “pharmacologically acceptable salts,” see Berge et al. J. Pharm. Sci. 66: 1, 1977. In certain embodiments of the invention salts of hydrochloride and salts of methanesulfonic acid can be used.

For administration, the compounds useful in this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds useful in this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, suppositories, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

The therapeutically effective amount of the calcium receptor-active compound in the compositions useful in the invention can range from about 0.1 mg to about 180 mg, for example from about 5 mg to about 180 mg, or from about 1 mg to about 100 mg of the calcimimetic compound per subject. In some aspects, the therapeutically effective amount of calcium receptor-active compound in the composition can be chosen from about 0.1 mg, about 1 mg, 5 mg, about 15 mg, about 20 mg, about 30 mg, about 50 mg, about 60 mg, about 75 mg, about 90 mg, about 120 mg, about 150 mg, about 180 mg.

While it may be possible to administer a calcium receptor-active compound to a subject alone, the compound administered will normally be present as an active ingredient in a pharmaceutical composition. Thus, a pharmaceutical composition of the invention may comprise a therapeutically effective amount of at least one calcimimetic compound, or an effective dosage amount of at least one calcimimetic compound.

As used herein, an “effective dosage amount” is an amount that provides a therapeutically effective amount of the calcium receptor-active compound when provided as a single dose, in multiple doses, or as a partial dose. Thus, an effective dosage amount of the calcium receptor-active compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multidose pharmaceutical composition, such as powders, liquids and the like, in which an effective amount of the calcimimetic compound is administered by administering a portion of the composition.

Alternatively, a pharmaceutical composition in which two or more unit dosages, such as in tablets, capsules and the like, are required to administer an effective amount of the calcium receptor-active compound may be administered in less than an effective amount for one or more periods of time (e.g., a once-a-day administration, and a twice-a-day administration), for example to ascertain the effective dose for an individual subject, to desensitize an individual subject to potential side effects, to permit effective dosing readjustment or depletion of one or more other therapeutics administered to an individual subject, and/or the like.

The effective dosage amount of the pharmaceutical composition useful in the invention can range from about 1 mg to about 360 mg from a unit dosage form, for example about 5 mg, about 15 mg, about 30 mg, about 50 mg, about 60 mg, about 75 mg, about 90 mg, about 120 mg, about 150 mg, about 180 mg, about 210 mg, about 240 mg, about 300 mg, or about 360 mg from a unit dosage form.

In some aspects of the present invention, the compositions disclosed herein comprise a therapeutically effective amount of a calcium receptor-active compound for the treatment or prevention of hyperacidic disorders. For example, in certain embodiments, the calcilytic compound can be present in an amount ranging from about 1% to about 70%, such as from about 5% to about 40%, from about 10% to about 30%, or from about 15% to about 20%, by weight relative to the total weight of the composition.

The compositions useful in the invention may contain one or more active ingredients in addition to the calcium sensing receptor-active compound. The additional active ingredient may be another calcilytic compound, or another calcimimetic compound, or it may be an active ingredient having a different therapeutic activity. Examples of such additional active ingredients include vitamins and their analogs, such as antibiotics, lanthanum carbonate, anti-inflammatory agents (steroidal and non-steroidal) and inhibitors of pro-inflammatory cytokine (ENBREL®, KINERET®). When administered as a combination, the therapeutic agents can be formulated as separate compositions that are given at the same time or different times, or the therapeutic agents can be given as a single composition.

In one aspect, the pharmaceutical compositions useful for methods of the invention may include additional compounds as described in more detail below. The term “combination therapy”, as used herein, is a therapy in which at least two active compounds in effective amounts are used to treat one or more of the disease states or conditions at the same time. The term “co-administration” describes the administration of two or more active compounds to the patient when effective amounts of the individual compounds are present in the patient at the same time. In one aspect, the compounds may be administered at the same time. The active compounds useful in the present invention include calcilytic compounds and additional compounds such as calcimimetics, proton pump inhibitors, H₂ blockers, antibiotics/antimicrobial agents, cytoprotective agents or other compounds in effective amounts for the disease or condition for which these compounds are typically used. These compounds are described in more detail in the section “Methods of treatment” below.

In another aspect, the compounds used to practice the methods of the instant invention can be formulated for oral administration that release biologically active ingredients. Upon ingestion, most acid-labile pharmaceutical compounds must be protected from contact with acidic stomach secretions to maintain their pharmaceutical activity. The term “acid-labile” compound or agent, is used herein to any pharmacologically active drug subject to acid catalyzed degradation.

In one aspect, the compositions of the instant invention may have enteric coating to dissolve at a certain pH. “Enteric coating,” as used herein, refers to a substance that remains substantially intact in the stomach but dissolves and releases the drug once the small intestine is reached. Generally, the enteric coating comprises a polymeric material that prevents release at the low pH but ionizes at a slightly higher pH, and thus dissolves sufficiently in the small intestine to gradually release the active agent. In one aspect, the compounds of the invention may be released in the proximal region of the small intestine (duodenum).

In another aspect, the compounds of the invention can be formulated in non-enteric coated pharmaceutical compositions. These compositions involve the administration of the compounds of the invention together with one or more buffering agents to allow for the immediate release of the pharmaceutically active ingredient. The buffering agent is intended to prevent substantial degradation of the pharmaceutical agent in the acidic environment of the stomach by raising the pH. See, e.g., U.S. Pat. Nos. 5,840,737; 6,489,346; 6,645,988 and 6,699,885.

In a further aspect of the invention, the compounds useful in the present invention, can be delivered to the stomach using floating drug delivery systems (FDDS). FDDS have a bulk density less than gastric fluids and thus remain buoyant in the stomach without affecting the gastric emptying rate for a prolonged period of time. While the system is floating on the gastric contents, the compounds of the invention are released slowly at the desired rate. After release of drug, the residual system is emptied from the stomach, thus resulting in an increased gastric retention time (GRT) and a better control of the fluctuations in plasma drug concentration. FDDS useful in the instant invention can be further divided into gas-generating and non-effervescent systems.

Gas-generating systems utilize matrices prepared with swellable polymers like methocel, polysaccharides such as chitosan, effervescent components such as sodium bicarbonate, citric acid and tartaric acid or chambers containing a liquid that gasifies at body temperature. The stoichiometric ratio of citric acid and sodium bicarbonate optimal for gas generation is 0.76:1. The common approach for preparing these systems involves resin beads loaded with bicarbonate and coated with ethyl cellulose. The insoluble coating allows permeation of water causing carbon dioxide to release and the beads to float in the stomach. Other approaches include the use of highly swellable hydrocolloids and light mineral oils, a mixture of sodium alginate and sodium bicarbonate, multiple unit floating pills that generate carbon dioxide when ingested, floating minicapsules with a core of sodium bicarbonate, lacotes and polyvinyl pyrrolodone coats with hydroxypropyl methylcellulose, and floating systems based on ion exchange resin technology.

Non-effervescent drug delivery systems useful in this invention after swallowing swell unrestrained via imbibitions of gastric fluid to an extent that it prevents their exit from the stomach. These systems also sometimes referred to as the “plug-type” systems since they have a tendency to remain lodged near the pyloric sphincter. To deliver the correct dose, the compounds useful in the present invention may be mixed with a gel, which swells in contact with gastric fluid after oral administration and maintains a relative integrity of shape and a bulk density of less than one within the outer gelatinous barrier. The air trapped by the swollen polymer confers buoyancy to this system. Other hydrodynamically balanced systems useful in the invention contain a mixture of compounds of the invention and hydrocolloids, sustained release capsules containing cellulose derivatives like starch and a higher fatty alcohol or fatty acid glyceride, bilayer compressed capsules, multilayered flexible sheet-like medicament devices, hollow microspheres of acrylic resins, polystyrene floatable shells, single and multiple unit devices with floatation chambers and mictoporous compartments and buoyant controlled release powder formulations. Other developments include use of superporous hydrogels that expand dramatically (hundreds of times their dehydrated dorm within seconds) when immersed in water. Oral drug delivery formulations made from the gels swell rapidly in the stomach, causing medications to move more slowly from the stomach to the intestines and be absorbed more efficiently by the body.

In one aspect of the invention, the calcilytic compounds useful in the present invention can be delivered using bioadhesive drug delivery systems (BDDS) that are used to localize a delivery device within the lumen to enhance the drug absorption in a site-specific manner. This approach involves the use of bioadhesive polymers, which can adhere to the epithelial surface in the stomach. See Chickering, D. E. et al. (1995) Reactive Polymers 25, 189-206. Excipients that can be used in these systems include polycarbophil, carbopol, lectins, chitosan, CMC and gliadin, as well as a novel adhesive material derived from the fimbriae bacteria or synthetic analogues combined with a drug to provide for attachment to the gut, thereby prolonging the transit time. Compositions comprising a calcilytic compound and a material that acts as a viscogenic agent, such as curdlan and/or a low-substituted hydroxypropylcellulose, are also useful in the present invention.

In another aspect of the invention, the calcilytic compounds can be delivered using sedimentation as a retention mechanism for pellets that are small enough to be retained in the rugae or folds of the stomach near the pyloric region. Dense pellets (approximately 3 g/cm³) trapped in rugae tend to withstand the peristaltic movements of the stomach wall. With pellets, the GI transit time can be extended from an average of 5.8 hours to 25 hours, depending more on density than on diameter of the pellets. Excipients such as barium sulphate, zinc oxide, titanium dioxide and iron powder increase density up to 1.5-2.4 g/cm³.

The calcilytic compounds useful in the present invention can be delivered using size-increasing drug delivery systems, such as unfolding multilayer, polymeric films based on a drug-containing shellac matrix as the inner layer, covered on both sides with outer shielding layers composed of hydrolyzed gelatin. See Klausner E. A. et al. (2002) Pharm. Res. 19: 1516-1523. This approach to retain a pharmaceutical dosage from in the stomach is based on increasing its size above the diameter of the pylorus. Another aspect of the invention deals with administering calcilytic compounds useful in the methods of this invention in enzyme-digestible hydrogels consisting of polyvinylpyrrolidone cross-linked with albumin. Shalaby WSW et al. (1992) J Control Release 19: 131-144. These hydrogels swell to a significant extent, which is a function of the albumin content and degree of albumin alkylation. The polymers are degraded in the presence of pepsin either via bulk or surface erosion. With increasing albumin alkylation, pepsin digestion is diminished and bulk erosion becomes predominant.

III. Methods of Treatment

In one aspect, the invention provides methods for treatment of hyperacidic disorders. Initial treatment of a subject suffering from a hyperacidic disease or disorder can begin with the dosages indicated above. Treatment is generally continued as necessary over a period of hours, days, weeks to months, or years until the disease or disorder has been controlled or eliminated. Subjects undergoing treatment with the compounds and compositions disclosed herein can be routinely monitored by any of the methods well known in the art to determine the effectiveness of therapy. Some of these methods are described in more detail below. Continuous analysis of such data permits modification of the treatment regimen during therapy so that optimal effective amounts of compounds of the instant invention are administered at any point in time, and so that the duration of treatment can be determined as well. Towards this goal, the treatment regimen and dosing schedule can be rationally modified over the course of therapy so that the lowest amount of a calcilytic compound is administered, and so that administration is continued only as long as necessary to successfully treat the disease or disorder.

Hyperacidic gastrointestinal disorders include, e.g., gastroesophageal reflux disease, non-erosive reflux disease, duodenal ulcer disease, gastrointestinal ulcer disease, erosive esophagitis, poorly responsive symptomatic gastroesophageal reflux disease, pathological gastrointestinal hypersecretory disease, Zollinger Ellison Syndrome, acid dyspepsia, heartburn, chronic hyperacidic gastritis, and duodenogastric reflux.

The invention provides methods for treatment of GERD in a variety of subjects. Certain medical and surgical conditions can predispose a person to GERD. The most common is pregnancy: 30 to 50% of pregnant women complain of heartburn, especially in the first trimester. Up to 90% of patients with scleroderma have GERD as the result of smooth muscle fibrosis causing low LES (lower esophageal sphincter) pressure and weak or absent peristalsis. Further, the methods described herein are useful for treating hyperacidic disorders in patients with the Zollinger-Ellison syndrome. In these patients, hypersecretion of acid and increased gastric volume are the major factors causing GERD. After Heller myotomy, 10 to 20% of patients may develop GERD. Finally, prolonged nasogastric tube intubation may contribute to the development of reflux esophagitis, in part because acid tracks along the tube and because the tube mechanically interferes with LES barrier function.

Besides being used for human treatment, the present invention is also useful for other subjects including veterinary, exotic and farm animals, including mammals such as primates, dogs, pigs, horses, cats, and rodents including rats, mice, or guinea pigs.

In one aspect, the hyperacidic disorders treated by the methods of the invention include gastroesophageal reflux disease (GERD, or acid reflux). The term GERD is a condition that occurs when the muscle between the esophagus and the stomach (lower esophageal sphincter) becomes or is weak or relaxes when it should not leading to the persistent return of stomach contents backwards up into the esophagus, frequently causing heartburn, a symptom of irritation of the esophagus by stomach acid. GERD results from the failure of the normal antireflux mechanism to protect against frequent and abnormal amounts of gastroesophageal reflux (GER), that is, the effortless movement of gastric contents from the stomach to the esophagus. GERD is a spectrum of disease usually producing symptoms of heartburn or acid regurgitation. Most patients have no visible mucosal injury at the time of endoscopic examination (non-erosive GERD), whereas others have esophagitis, peptic strictures, Barrett esophagus, or evidence of extraesophageal diseases such as chest pain, pulmonary symptoms, or ear, nose, and throat symptoms. The pathophysiology of GERD is complex and results from an imbalance between defensive factors protecting the esophagus, such as antireflux barriers, esophageal acid clearance, tissue resistance, and aggressive factors from the stomach contents, such as gastric acidity and volume and duodenal contents. The aggressive factors and defensive forces are part of delicately balanced system.

One of the classic symptoms of GERD is heartburn, with patients generally reporting a burning feeling, rising from the stomach or lower chest and radiating toward the neck, throat and occasionally the back. Usually it occurs postprandially, particularly after large meals or the consumption of spicy foods, citrus products, fats, chocolates, and alcohol. The diagnosis of GERD usually is based on the occurrence of heartburn on two or more days a week, also less frequent symptoms do not preclude the disease. However, the frequency and severity of heartburn do not predict the degree of esophageal damage. Other common symptoms of

GERD are acid regurgitation and dysphagia. The effortless regurgitation of acidic fluid, especially after meals and exacerbated by stooping or recumbency, is highly suggestive of GERD. Among patients with daily regurgitation, the LES pressure usually is low, many have associated gastroparesis, and esophagitis is common. Dysphagia is reported by more than 30% of patients with GERD. It usually occurs in the setting of long-standing heartburn, with slow progressive dysphagia primarily for solids. Less common reflux-associated symptoms include water brash (the sudden appearance in the mouth of a slightly sour or salty fluid), odynophagia (pain on swallowing), burping, hiccups, nausea, and vomiting. Further, some patients with GERD are asymptomatic, especially elderly patients because of decrease acidity of the reflux material or decreased pain perception. Extraesophageal manifestations of GERD may include non-cardiac chest pain (described as squeezing or burning, substernal in location, and radiating to the back, neck, jaw, or arm), asthma, posterior laryngitis, chronic cough, recurrent pneumonitis, and dental erosion.

While the classic symptoms of heartburn and acid regurgitation are sufficiently specific to identify reflux disease and begin medical treatment, a clinician may use a reliable and cost-effective test for evaluating patients with suspected GERD. In one aspect, the empirical trial of acid suppression may be used. The initial dose of proton pump inhibitor or PPI (e.g., omeprazole 40 to 80 mg/day) can be given for not less than 14 days. If symptoms disappear with therapy and then return when the medication is stopped, GERD may be assumed. Upper endoscopy is the current standard for documenting the type and extent of mucosal injury to the esophagus. It identifies the presence of esophagitis and excludes other causes of patient's complaints. However, only 40 to 60% of patients with abnormal esophageal reflux by pH testing have endoscopic evidence of esophagitis. The earliest endoscopic signs of acid reflux include edema and erythema. Other findings include friability (easy bleeding), granularity and red streaks. With progressive acid injury, erosions develop. Endoscopic grading of GERD depends on the endoscopist's interpretation of these visual images. On of the most popular grading systems used in the United States is the Los Angeles system, wherein the number, length, and location of mucosal breaks determine the degree of esophagitis (Table 1).

TABLE 1
Los Angeles Classification for Esophagitis

| | |
|---|---|
| Grade A | One ore more mucosal beaks confined to folds, no longer |
| | than 5 mm |
| Grade B | One or more mucosal breaks >5 mm confined to folds but |
| | not continuous between tops of mucosal folds |
| Grade C | Mucosal breaks continuous between tops of two or more |
| | mucosal folds but not circumferential |
| Grade D | Circumferential mucosal break |

Biopsies of the esophagus help to identify reflux injury, exclude other esophageal diseases, and confirm the presence of complications, especially Barrett esophagus. Microscopic changes indicative of reflux may occur even when the mucosa appears normal endoscopically. The most sensitive histological markers of GERD are reactive epithelial changes characterized by an increase in the basal cell layer greater than 15% of the epithelium thickness or papilla elongation into the upper third of the epithelium. These changes represent increased epithelial turnover of the squamous mucosa. Acute inflammation characterized by the presence of neutrophils and eosinophils is very specific for esophagitis.

Ambulatory intraesophageal pH monitoring is now the standard for establishing pathological reflux. The test is performed with a pH probe passed nasally and positioned 5 cm above the manometrically determined LES. Monitoring is usually carried out for 18 to 24 hours. Reflux episodes are detected by a drop in pH to less than 4. Commonly measured parameters include the percentage of total time that the pH is less than 4, the percentage of time upright and supine that the pH is less than 4, the total number of reflux episodes, the duration of longest reflux episode, and the number of episodes longer than 5 minutes. The total percentage of time that the pH is less than 4 is the most reproducible measurement for GERD, with reported upper limits of normal values ranging from 4% to 5.5%. Kahrilas P. J. et al. (1996) Gastroenterology 110: 1982.

Another inexpensive and noninvasive test helpful to establish a presence of a hyperacidic disorder such as GERD in a patient is barium esophagram. It is very useful in demonstrating structural narrowing of the esophagus and in assessing the presence and reducibility of a hiatal hernia. This test is used in evaluating the patient with GERD with new-onset dysphagia because it can define subtle strictures and rings as well as assess motility.

Esophageal manometry allows accurate assessment of LES pressure and relaxation, as well as peristaltic activity including contraction amplitude, duration and velocity. Radiolabeled technetium-99m sulfur colloid scintiscanning is useful as a semiquantitative test for detecting GER. The acid perfusion (Bernstein) test is useful for detecting the relationship of symptoms to esophageal acidification. Bile reflux can be measured using ambulatory esophageal bilirubin monitoring.

Other hyperacidic disorders that can be treated using the methods described in the present invention include non-erosive reflux disease, erosive reflux disease and various complications of the disorders described below. The present invention may be used to treat these disorders in a patient by administering an effective amount of a calcilytic compound, either alone or in combination with at least one other of the traditional treatment modalities known in the art, described in more detail below.

Non-erosive GERD, non-erosive reflux disease, or NERD is used to describe a specific form of reflux disease that is characterized by the presence of typical symptoms of GERD due to intraesophageal acid in the absence of visible esophageal mucosal injury on endoscopy. NERD is suspected by the presence of typical reflux symptoms with a normal endoscopic examination and is confirmed by the patient's response to antisecretory therapy. Overall, patients with non-erosive reflux disease do not respond to antireflux therapy as well as patients with erosive GERD. Fass R. et al. (2001) Am. J. Gastroenterol. 96: 303.

Zollinger-Ellison or ZE syndrome is a condition caused by abnormal production of the hormone gastrin. In this disorder, small tumor (gastrinoma) in the pancreas or small intestine produces the high level of gastrin in the blood, which causes overproduction of stomach acid. In turn, high stomach acid levels lead to multiple ulcers in the stomach and small bowel.

The methods of this invention may be used to treat ulcer. “Ulcer” means an area of tissue erosion , especially of the lining of the gastrointestinal tract, such as stomach (peptic ulcer), esophagus or small intestine (duodenal ulcer). Ulcers are always depressed below the level of the surrounding tissue. They can have diverse causes, but in the GI tract they are believed to be primarily due to infection with the bacteria H. piloridus (h. pilori). The present invention may be used to treat an H. piloridus infection in a patient by administering an effective amount of a calcilytic compound, either alone or in combination with at least one other of the traditional treatment modalities known in the art.

The methods of the invention described herein may be useful in treating various complications of the hyperacidic disorders. For example, hemorrhage and esophageal perforation are complications of reflux esophagitis and are usually associated with deep esophageal ulcers or severe diffuse esophagitis. While esophageal perforations are very rare, they can result in mediastinitis and can be fatal if they are not rapidly recognized and treated. Peptic esophageal stricture occurs in patients with untreated reflux esophagitis, especially in older men. They usually evolve over many years and may be linked to the long-term use of non-steroidal anti-inflammatory drugs. In some patients with GERD, the squamous epithelial of the distal esophagus is replaced by specialized columnar epithelium, resembling that of the intestine and containing goblet cells. These patients with a disorder called Barrett esophagus have severe GERD with low LES pressure, poor esophageal motility, large hiatal hernias, and extensive acid and bile reflux, wherein most patients have had chronic reflux symptoms for at least 10 years.

The methods of the present invention can be used in combination with one or more of the traditional modalities known in the art. For example, in patients without esophagitis, the therapeutic goal may be to relieve the acid-related symptoms and to prevent frequent symptomatic relapses. In patients with esophagitis, the goals are to relieve symptoms and to heal the esophagitis while attempting to prevent further relapses and the development of complications.

In one aspect, the methods of the present invention can be practiced together with lifestyle modifications. These include head of bed elevation, avoidance of tight-fitting clothes, weight loss, restriction of alcohol, elimination of smoking, dietary therapy, refraining from lying down after meals, and avoidance of evening snacks before bedtime. These changes may be especially recommended for patients with nocturnal GERD symptoms or laryngeal complaints.

In another aspect, calcilytic compounds of the invention can be co-administered with other therapeutic compounds. The active compositions may include one ore more calcilytic compounds and additional compounds or compositions such as antacids, Gaviscon, prokinetics, H₂ receptor antagonists, proton pump inhibitors, antibiotics/antimicrobial agents, cytoprotective agents or combination agents in effective amounts for the hyperacidic disease or disorder for which the compounds are typically used. For example, calcilytic compounds of the invention can be used in combination with antacids, such as Mylanta, Maalox, or Riopan. Antacids increase LED pressure but work primarily by buffering gastric acid in the esophagus and stomach for relatively short periods. In another example, the compounds of the invention can be co-administered with Gaviscon, which mixes with saliva to form a highly viscous solution that floats on the surface of the gastric pool and acts as a mechanical barrier. In another aspect, the compounds and compositions of the invention can be co-administered with prokinetic drugs, which improve reflux symptoms by increasing LES pressure, acid clearance, or gastric emptying. The examples of prokinetics include bethanechol (Urecholine), metoclopramide (Reglan), domperidone and cisapride, a serotonin receptor agonist. In a further aspect, the compounds and compositions of the invention can be co-administered with Histamine₂ (H₂) receptor antagonists. They are most effective in controlling nocturnal, as compared with meal-related acid secretion. H₂ receptor antagonists include cimetidine (Tagamet), ranitidine (Zantac), famotidine (Pepcid), and nizatidine (Axid). In another aspect, the compounds and compositions of the invention can be co-administered with proton pump inhibitors (PPIs), which diminish gastric acid secretion by inhibiting the final common pathway of acid secretion, the H⁺, K⁺-ATPase pump. The examples of PPIs include omeprazole (Prilosec), lansoprazole (Prevacid), rabeprazole (Aciphex), pantoprazole (Protonix), and esomeprazole (Nexium).

In one aspect, the methods of the invention can be practiced in combination with surgical treatment, or endoscopic treatment, such as endoscopic suturing systems, radiofrequency energy delivery to the gastroesophageal junction, and the injection of non-absorbable polymers into the submucosa surrounding the LES.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The following examples are offered to more fully illustrate the invention, but are not to be construed as limiting the scope thereof.

### EXAMPLE 1

This example outlines methods and techniques used in the present invention.

Animals.

Male (Casr^{+/+}; Gcm2^{-/-}) or CaSR knockout (Casr^{-/-}; Gcm2^{-/-}) mice weighing 22-27 grams (upper panel) or male Sprague-Dawley rats weighing 220-275 grams (lower panel) were fasted with ad lib access to water for 18 hours prior to experimentation. The animals were exposed to an overdose of isofluorane and the stomach was removed by a total gastrectomy. The corpus was removed from the stomach and sectioned at 4° C. All mice were generated at Yale University from a breeding colony. Male Sprague-Dawley rats were purchased from Charles River Laboratories Inc. (Wilmington, Mass.). All animals were cared for according to the standard protocols of the Yale University Animal Care and Use Committee.

Chemical Reagents.

The HEPES-Ringer solution contained (in mmol/L): NaCl 125; KCl 5; MgCl₂ 0.5; HEPES 22, CaCl₂ 0.1 or 1.6; glucose 10, pH=7.4. The solution was bubbled with 100% O₂. 2′,7′-bis′(carboxyethyl)-5-(6)-carboxyfluorescein (BCECF) from Invitrogen (Seattle, Wash., USA) and stock solutions were prepared in dimethyl sulphoxide (DMSO).

Calcimimetic solution (Compound A, 3-(2-chlorophenyl)-N-(1-(3-(methyloxy)phenyl)ethyl)-1-propanamine, and the calcilytic solution, Compound B, 2-chloro-6-(2-hydroxy-3-(2-methyl-1-(naphthalen-2-yl)propan-2-ylamino)propoxy)benzonitrile, were dissolved in DMSO. Final concentrations of DMSO never exceeded 0.1% (v/v). Preliminary experiments indicated that the vehicle did not alter any baseline electrophysiological parameters.

Gland Dissection and pH-Sensing Dye Loading.

Individual gastric glands were hand dissected and transferred to the stage of an inverted microscope where they were loaded with the intracellular pH marker BCECF. Once loaded with the dye the glands were superfused with normal HEPES Ringer solution at 37° C. and at a pH of 7.4 for 5 minutes. The glands were then challenged with 20 mM NH₄Cl and 0 mM Na to induce an acid load within the cell. The rate of recover was then calculated in the presence or absence of the calcimimetic Compound A at either 10 or 100 nM concentration.

Statistical Analysis.

The rate of recovery in the absence of Na was recorded for each parietal cell and then summated and the mean ±SEM of the data for each gland and then for each animal determined. The final rate of recovery shows the number of animals with a minimum of 5 glands per animal and 5-7 cells per gland. The recovery rates are determined as the rate of recovery following an acid load. This rate determines how fast the cell can extrude protons and provides a measure of the activity of the gastric H,K-ATPase proton pump.

### EXAMPLE 2

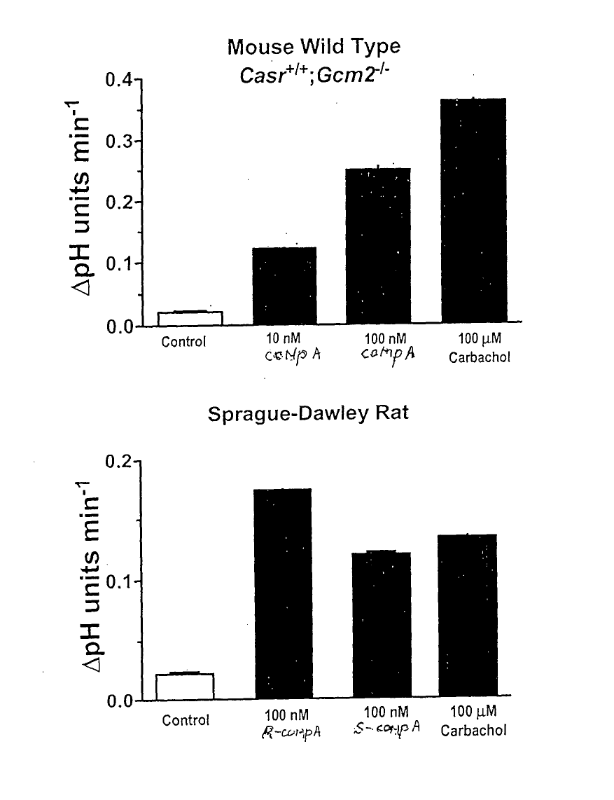

This experiment (FIG. 1) demonstrates the ability of addition of a calcimimetic to induce acid secretion by superfused gastric glands isolated either from control Casr^{+/+}; Gcm2^{-/-} mice (upper panel) or Sprague-Dawley rats (lower panel). Rates of recovery were compared to 100 μM concentration of the cholinergic, carbachol, a secretagogue used to maximally stimulate gastric acid secretion by gastric glands. The calcimimetic Compound A increased acid secretion by gastric glands from both mice and rats. The effect of Compound A was concentration dependent and the S enantiomer was less potent than the R enantiomer. The data demonstrate the ability of a calcimimetic to stimulate gastric acid secretion.

### EXAMPLE 3

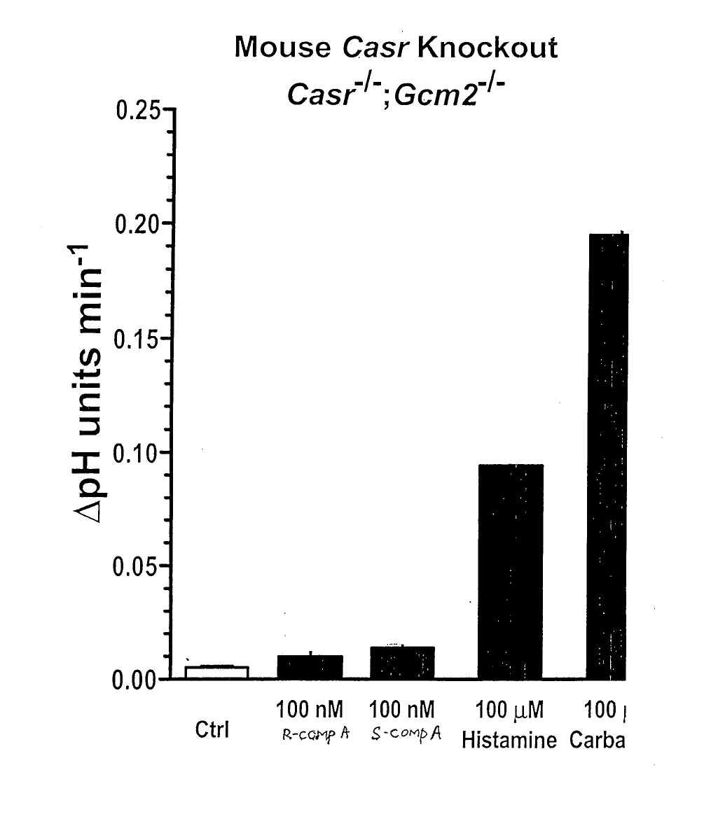

This experiment (FIG. 2) demonstrates that the effect of a calcimimetic to stimulate gastric acid secretion requires expression of a functional CaSR in gastric parietal cells. Gastric acid secretion is measured in Casr^{-/-}; Gcm2^{-/-} mice that lack a functional CaSR. 100 μM histamine or 100 μM carbachol increases acid secretion demonstrating that mice lacking a functional CaSR exhibit a normal capacity to secrete acid when stimulated by natural secretagogues. This demonstrates that CaSR knockout mice have the cellular machinery for the classical secretagogue pathways to stimulate acid secretion. In contrast, neither R nor S enantiomer of Compound A has an effect on basal acid secretion in these CaSR knockout mice demonstrating that the action of a calcimimetic to increase gastric acid secretion requires the presence of a functional CaSR.

### EXAMPLE 4

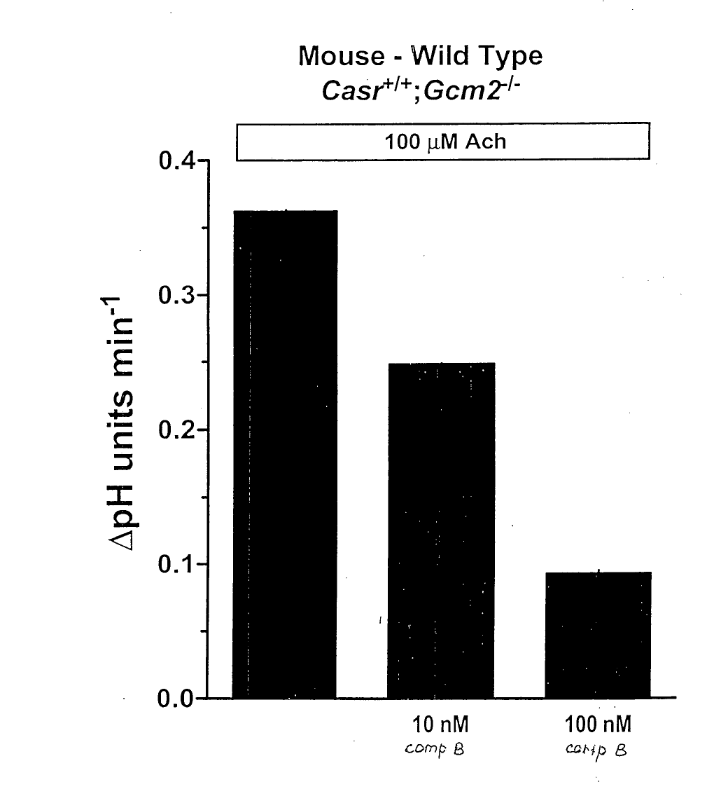

This experiment (FIG. 3) demonstrates the effect of the calcilytic Compound B to inhibit secretagogue induced acid secretion. The mouse gastric glands were exposed to a 100 μM acetyl choline (AcH), a potent activator of acid secretion, prior to and during the acid challenge generated with 20 mM NH₄Cl and 0 mM Na to induce an acid load within the cell. The rate of recovery was then calculated in the presence or absence of the calcilytic compound B at either 10 or 100 nM concentration. Addition of the calcilytic to animals with a functional CaSR resulted in an inhibition of acid secretion in a concentration dependent fashion.

### EXAMPLE 5

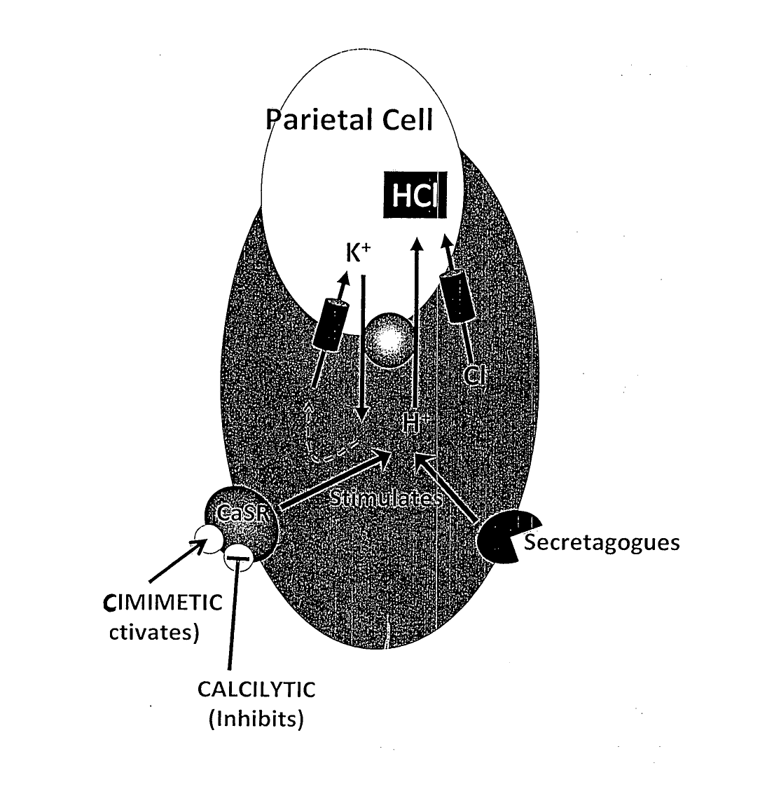

This experiment (FIG. 4) demonstrates the effect of the calcilytic Compound B on inhibiting acid secretion in mice with a constitutively active H,K-ATPase. These mice have a mutation in the gastric H,K-ATPase proton pump that renders the pump constitutively active. Addition of the calcilytic to these mice suppresses gastric acid secretion in a concentration-dependent fashion.

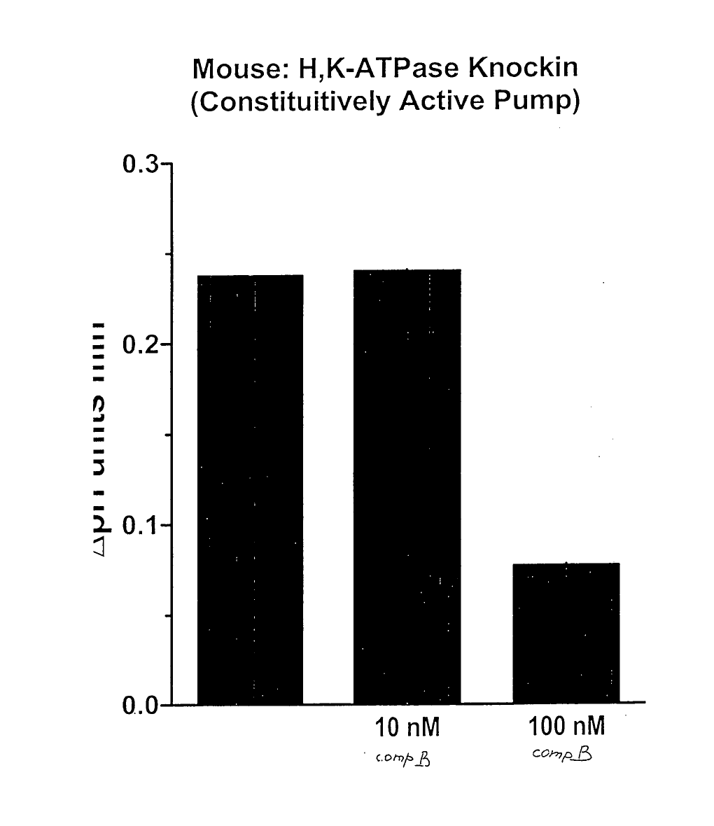

FIG. 5 illustrates the dose-dependent effect of calcilytic Compound B to reduce acid secretion by the superfused gastric gland isolated from mice that express the functional calcium sensing receptor and have a constitutively active gastric H,K-ATPase proton pump.

### EXAMPLE 6

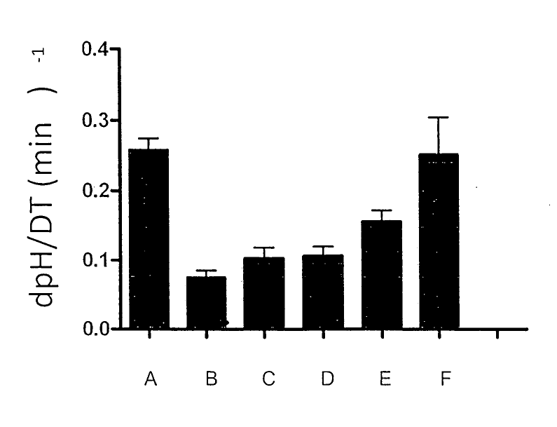

This experiment (FIG. 6) demonstrates that when cells are activated first by a hormonal secretagogue, such as released after a meal, the addition of a calcimimetic can inhibit acid secretion as demonstrated in superfused gastric glands isolated from Sprague-Dawley rats.

Rates of recovery were compared to 200 μM concentration of the hormonal secretagogue histamine (bar A) used to maximally stimulate gastric acid secretion by gastric glands. When histamine was added alone (bar A) there was an activation of acid secretion. The calcimimetic Compound A decreased acid secretion in gastric glands from rats treated with a secretagogue (bars B, C, D). The effect of calcimimetic Compound A when added in the absence of a secretagogue induced acid secretion (bars E, F). The summary data from 5 rats, 4 glands per rat, and 10 cells per gland demonstrate the ability of a calcimimetic to decrease secretagogue induced acid secretion.

What is claimed is:

1. A method for treatment of a hyperacidic disease or disorder comprising administering an effective amount of a calcilytic compound or a pharmaceutically acceptable salt thereof to a subject in need thereof.

2. The method of claim 1, wherein the hyperacidic disorder is GERD.

3. The method of claim 1, wherein the hyperacidic disorder is NERD.

4. The method of claim 3, wherein the hyperacidic disease is peptic esophageal strictures, Barrett esophagus, or gastric adenocarcinoma.

5. The method of claim 1, wherein GERD is mild, moderate or severe.

6. The method of claim 1, wherein the hyperacidic disorder is caused by an Helicobacter pylori colonization, hiatus hernia, gastritis, active duodenal ulcers, gastric ulcers, Zollinger-Ellison syndrome, dyspepsia, duodenogastric reflux, or delayed gastric emptying.

7. The method of claim 1, further comprising administering a compound for treating heartburn.

8. The method of claim 1, further comprising administering a compound for treating acid regurgitation.

9. The method of claim 1, further comprising administering a compound for treating dysphagia.

10. The method of claim 1, further comprising administering a compound for treating water brash, odynophagia, burping, hiccups, nausea, or vomiting.

11. The method of claim 1, further comprising administering a compound for treating non-cardiac chest pain, asthma, posterior laryngitis, reflux laryngitis, chronic cough, recurrent pneumonitis, or dental erosion.

12. The method of claim 1, further comprising administering an antacid.

13. The method of claim 1, further comprising administering a prokinetic agent.

14. The method of claim 1, further comprising administering an H₂ receptor antagonist.

15. The method of claim 1, further comprising administering a proton pump inhibitor.

16. The method of claim 1, further comprising maintenance therapy.

17. The method of claim 1, further comprising administering a calcimimetic compound.

18. The method of claim 1, wherein the subject is human.

19. The method of claim 18, wherein the subject is elderly or pregnant.

20. The method of claim 1, wherein the calcilytic compound is 2-chloro-6-(2-hydroxy-3-(2-methyl-1-(naphthalen-2-yl)propan-2-ylamino)propoxy)benzonitrile.

21. A method for treatment of a hyperacidic disorder comprising administering an effective amount of a calcimimetic compound or a pharmaceutically acceptable salt thereof in combination with a PPI to a subject in need thereof.
